(19) United States
(12) Patent Application Publication
(10) Pub. No.: US 2016/0274023 A1
(43) Pub. Date: Sep. 22, 2016

(54) Quantum Cascading Laser System for Analyzing Pathogenic Microorganisms

(71) Applicant: Daniel L. Carpenter, Memphis, TN (US)

(72) Inventor: Daniel L. Carpenter, Memphis, TN (US)

(21) Appl. No.: 14/660,670

(22) Filed: Mar. 17, 2015

(51) Int. Cl. G01N 21/39; G01N 33/49

(52) U.S. Cl. CPC G01N 21/39; G01N 33/492; G01N 2021/396; G01N 2201/06113

(57) ABSTRACT

A quantum cascading laser system for analyzing pathogenic microorganisms, where the quantum cascading laser system comprises a tunable laser unit, a beam splitter, lens, sample chamber, optical detector, data acquisition system, reference detector, and lock-in amplifier, where a computer controls the system and conducts the data analysis from the data produced from the quantum cascading laser system against a set of data derived from known pathogenic mircoorganisms to identify the pathogenic organisms in the original sample.

### CROSS-REFERENCE TO RELATED APPLICATIONS

| | | |
|---|---|---|
| 8,841,118 | September 2014 | Robinson et al. |
| 8,835,128 | September 2014 | Culha |
| 8,787,633 | July 2014 | Robinson et al. |
| 8,780,347 | July 2014 | Kotidis et al. |
| 8,748,122 | June 2014 | Hyman et al. |
| 8,723,106 | May 2014 | van Wuijckhuijse et al. |
| 8,709,748 | April 2014 | Walsh et al. |
| 8,688,348 | April 2014 | Puppels et al. |
| 8,655,807 | February 2014 | Multari et al. |
| 8,502,148 | August 2013 | Wagner et al. |
| WO2013093913 | June 2013 | Gannot et al. |
| 2011/0205524 | August 2011 | Puzey |
| 7,894,057 | February 2011 | Puzey |
| 7,436,510 | October 2008 | Grun et al. |
| 7,262,840 | August 2007 | Maier et al. |
| 7,256,875 | August 2007 | Maier et al. |
| 6,917,423 | July 2005 | Gardner, Jr. et al. |

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### FIELD OF THE INVENTION

This disclosure relates to the use of a quantum cascading laser system operating in an iterative process, for analyzing the constituents within blood such as pathogenic microorganisms including but not limited to bacteria, viruses, and fungi, blood composition including but not limited to glucose levels, cholesterol levels, insulin levels, and electrolytes, organic chemical compounds, inorganic chemical compounds including but not limited to explosive compounds, and elemental isotopes. Where the quantum cascading laser system comprises a tunable laser unit, a beam splitter, lens, sample chamber, optical detector, data acquisition system, reference detector, and lock-in amplifier. Where the computer controls the system and conducts the data analysis from the data produced from the quantum cascading laser system against a set of data derived from known constituents to identify the constituents in the original sample.

### BACKGROUND OF THE INVENTION

Pathogenic mircoorganisms impact our lives every day. Many of these organisms are harmless, yet others can cause serious, even life threatening diseases. Being able to rapidly identify pathogenic microorganisms and distinguish which are harmless from which are life threatening is very important for treatment as well as protecting the nation from incoming biologic threats. The current state of the art in identifying pathogenic microorganisms involves obtaining a blood sample and then culturing the sample until the pathogenic microorganisms are in sufficient quantity to be identified optically. This is a time consuming process that may take weeks. The present system is capable of using small amounts of blood to detect trace amounts of pathogenic microorganisms, in the process, reducing the amount of time to analyze a sample down to minutes, thus allowing treatment to begin sooner and alerts issued faster and is capable of analyzing for a wide array of blood components, including blood composition including but not limited to glucose levels, cholesterol levels, insulin levels, and electrolytes, organic chemical compounds, inorganic chemical compounds including but not limited to explosive compounds, and elemental isotopes.

Several attempts have been made to use quantum cascading laser system, all of which can be distinguished from the present disclosure:

In U.S. Pat. No. 8,841,118, Robinson et al., discloses a detection instrument to determine if a microbial agent is present, whereas the present disclosure characterizes the microbial agents present.

In U.S. Pat. No. 8,835,128, Culha, discloses a method for the identification of bacteria with surface enhanced raman scattering, whereas the present disclosure characterizes bacterial agents through the use of a quantum cascading laser system.

In U.S. Pat. No. 8,787,633, Robinson et al., discloses a system and method for identifying organisms by analysis of scattergrams of colonies, whereas the present disclosure reveals a method of characterizing bacterial agents in blood samples.

In U.S. Pat. No. 8,748,122, Hyman et al., discloses methods and systems for scanning, detecting, and monitoring microorganisms on solid or semi-solid media using intrinsic fluorescence (IF) measurements, whereas the present disclosure characterizes bacterial agents through the use of a quantum cascading laser system.

In U.S. Pat. No. 8,723,106, van Wuijckhuijse et al., discloses the characterization of biological material using matrix assisted laser desorption and ionization-mass spectroscopy, whereas the present disclosure reveals the characterization of bacterial agents through the use of a quantum cascading laser system.

In U.S. Pat. No. 8,709,748, Walsh et al., discloses a method for detecting, and characterizing a microorganism present in a sample and growth composition whereby the method may be accomplished utilizing a time-dependent spectroscopic technique to obtain at least two measurements directly from a sample and growth composition and correlating said measurements for the detection and characterization of a microorganism, whereas the present disclosure reveals the characterization of bacterial agents through the use of a quantum cascading laser system.

In U.S. Pat. No. 8,688,384, Puppels et al., discloses the use of Raman spectroscopy to characterize bacterial samples, whereas the present disclosure reveals the characterization of bacterial agents through the use of a quantum cascading laser system.

In U.S. Pat. No. 8,655,807, Multari et al., discloses a method for forming a recognition algorithm for laser-induced breakdown spectroscopy, whereas the present disclosure reveals the characterization of bacterial agents through the use of a quantum cascading laser system.

In U.S. Pat. No. 8,502,148, Wagner et al., discloses a method of characterization of a single particle with the use of a quantum cascading laser, whereas the present disclosure reveals the characterization of a homogenous sample without molecular separation.

In Patent No. WO2013093913, Gannot et al., discloses A spectroscopic method for detection and identification of bacteria in culture, whereas the present disclosure reveals the characterization of a homogenous sample through the use of a quantum cascading laser system.

In U.S. Pat. App. No. 2011/0205524, Puzey, discloses the use of broadband light, for example, from quantum cascade laser, to determine information about one or more substances in a sample, whereas the present disclosure reveals the characterization of a sample through the use of a quantum cascading laser in an iterative process.

In U.S. Pat. No. 7,894,057, Puzey, discloses the use of broadband light, for example, from quantum cascade laser, to determine information about one or more substances in a sample, whereas the present disclosure reveals the characterization of a sample through the use of a quantum cascading laser in an iterative process.

In U.S. Pat. No. 7,436,510, Grun et al., discloses a method for characterizing a sample using a laser and comparing the spectrographic results with a known database, whereas the present disclosure reveals the characterization of a sample through the use of a quantum cascading laser in an iterative process.

In U.S. Pat. No. 7,262,840, Maier et al., discloses a method for the detection and identification of pathogenic microorganisms using Raman scattered light and emitted light, whereas the present disclosure reveals the characterization of a sample through the use of a quantum cascading laser in an iterative process.

In U.S. Pat. No. 7,256,875, Maier et al., discloses a method for the detection and identification of pathogenic microorganisms using Raman scattered light and emitted light, whereas the present disclosure reveals the characterization of a sample through the use of a quantum cascading laser in an iterative process.

In U.S. Pat. No. 6,917,423, Gardner, discloses the characterization of pathogenic microorganisms by spectral imaging of the Raman light scattered by the organisms, whereas the present disclosure reveals the characterization of a sample through the use of a quantum cascading laser in an iterative process.

### SUMMARY OF THE INVENTION

This disclosure relates to the use of a quantum cascading laser system in an iterative process for analyzing the constituents in blood such as pathogenic microorganisms including but not limited to bacteria, viruses, and fungi, blood composition including but not limited to glucose levels, cholesterol levels, insulin levels, and electrolytes, organic chemical compounds, inorganic chemical compounds including but not limited to explosive compounds, and elemental isotopes.

The quantum cascading laser system comprises a tunable laser unit, a beam splitter, lens, sample chamber, optical detector, data acquisition system, reference detector, and lock-in amplifier. The computer controls the system in an iterative process in order to detect the constituents in the sample and conducts the data analysis from the data produced from the quantum cascading laser system with a set of data derived from known constituents. In the first embodiment, the quantum cascading laser system operates as a pulse or series of pulses. In a second embodiment, the quantum cascading laser system operates as a continuous beam.

### BRIEF DESCRIPTION OF THE SEVERAL VIEWS OF THE DRAWING

FIG. 1 is a line drawing displaying the flow of the quantum cascading laser system.

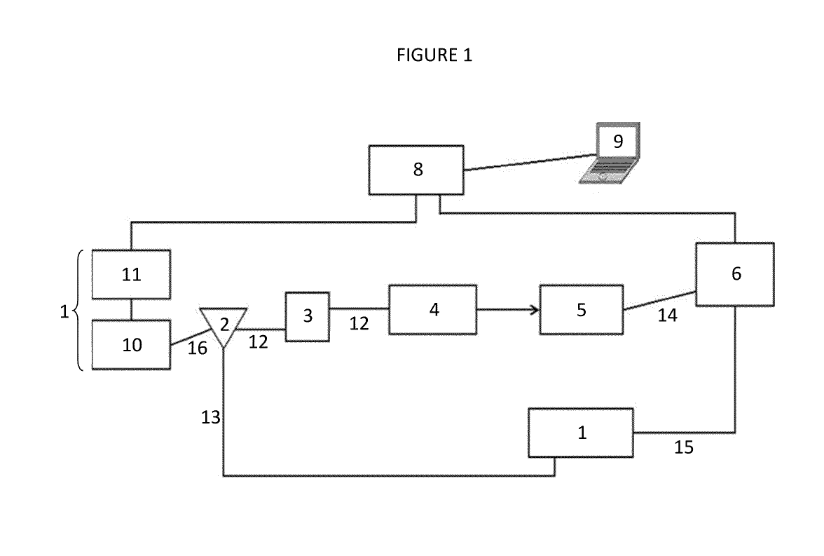

### DETAILED DESCRIPTION OF THE INVENTION

A pulsed quantum cascading laser system operating in an iterative process for analyzing blood constituents such as pathogenic microorganisms including but not limited to bacteria, viruses, and fungi, blood composition including but not limited to glucose levels, cholesterol levels, insulin levels, and electrolytes, organic chemical compounds, inorganic chemical compounds including but not limited to explosive compounds, and elemental isotopes comprising a tunable laser unit 1, a beam splitter 2, lens 3, sample chamber 4, optical detector 5, data acquisition system 6, reference detector 7, and lock-in amplifier 8, which is controlled by a computer 9.

The tunable laser unit comprises a quantum cascading laser 10 and a laser controller 11. The quantum cascading laser 10 generates a beam, the frequency and intensity of which is controlled by the laser controller 11. The beam exits the quantum cascading laser 10 and is received by the beam splitter 2, which divides the beam into two parts, part one of the beam 12 and part two of the beam 13. Part one of the beam 12 exits the beam splitter 2 and enters the lens 3 which orients the beam at the sample to be analyzed. The part 1 of the beam 12 then passes through the lens 3 and into the sample chamber 4 where it impacts the sample. A portion of part one of the beam 12 that impacts the sample is absorbed. After that portion of part one of the beam 12 is absorbed, the sample releases energy, producing an output absorbance wave. The non-absorbed portion of part one of the beam and output absorbance wave is then detected by the optical detector 5 which creates data set two 14, which is then sent to the data acquisition system 6.

Part two of the beam 13 exits the beam splitter 2 and enters the reference detector 7, which identifies the frequencies of part two of the beam 13, as well as the intensity of part two of the beam 13, creating data set three 15, which is fed into the data acquisition system 6.

The lock-in amplifier 8 receives the various information including the initial frequency and intensity of the beam that exits the quantum cascading laser, which is data set one 16, as well as data set two and data set three, to then identify the unabsorbed portion of part one of the beam 12, identify the frequency and intensity of the output absorbance wave, determine and separate out that portion of the output absorbance wave that is the result of the blood plasma and water, and characterize the remaining frequencies and intensities of the output absorbance wave, the spectra.

The computer 9 then receives the spectra and compares the frequencies and intensities of the spectra against a database of known spectra to characterize the pathogenic microorganisms and other constituents in the sample. The process continues in an iterative process and through various spectra from the pulsed quantum cascading laser system until the constituents in the blood are fully characterized.

What is claimed:

1. A pulsed quantum cascading laser system operating in an iterative process for analyzing the constituents in blood including pathogenic microorganisms including but not limited to bacteria, viruses, and fungi, blood composition including but not limited to glucose levels, cholesterol levels, insulin levels, and electrolytes, organic chemical compounds, inorganic chemical compounds including but not limited to explosive compounds, and elemental isotopes comprising:
A tunable laser unit, a beam splitter, lens, sample chamber, optical detector, data acquisition system, reference detector, and lock-in amplifier;
wherein the tunable laser unit comprises a quantum cascading laser and a laser controller;
wherein during each iteration of the pulsed quantum cascading laser system the quantum cascading laser generates a beam, the frequency and intensity of which is controlled by the laser controller;
wherein the beam exits the quantum cascading laser and is received by the beam splitter, which divides the beam into two parts, part one of the beam and part two of the beam;
wherein part one of the beam exits the beam splitter and enters the lens which orients the beam at the sample to be analyzed, passes through the lens and into the sample chamber where it impacts the sample;
wherein a portion of part one of the beam that impacts the sample is absorbed by the sample;
wherein after that portion of part one of the beam is absorbed, the sample releases energy, producing an output absorbance wave;
wherein the non-absorbed portion of part one of the beam and output absorbance wave is then detected by the optical detector which creates data set 2, which is then sent to the data acquisition system;
wherein part two of the beam exits the beam splitter and enters the reference detector, which identifies the frequencies of part two of the beam, as well as the intensity of part two of the beam, creating data set 3, which is fed into the data acquisition system;
wherein the lock-in amplifier receives the various information including the initial frequency and intensity of the beam that exits the quantum cascading laser, which is data set 1, as well as data set 3 and data set 2, to then identify the unabsorbed portion of part one of the beam, identify the frequency and intensity of the output absorbance wave, determine and separate out that portion of the output absorbance wave that is the result of the blood plasma and water, and characterize the remaining frequencies and intensities of the output absorbance wave, the spectra;
wherein the pulsed quantum cascading laser system is controlled by a computer; and
wherein a computer also receives the spectra from the and then conducts the comparison of the frequencies and intensities of the spectra with a database of known spectra to characterize the pathogenic microorganisms and other trace constituents in the sample.

2. A continuous quantum cascading laser system for analyzing pathogenic microorganisms comprising:
A tunable laser unit, a beam splitter, lens, sample chamber, optical detector, data acquisition system, reference detector, and lock-in amplifier;
wherein the tunable laser unit comprises a quantum cascading laser and a laser controller;
wherein during each iteration of the continuous quantum cascading laser system the quantum cascading laser generates a beam, the frequency and intensity of which is controlled by the laser controller;
wherein the beam exits the quantum cascading laser and is received by the beam splitter, which divides the beam into two parts, part one of the beam and part two of the beam;
wherein part one of the beam exits the beam splitter and enters the lens which orients the beam at the sample to be analyzed, passes through the lens and into the sample chamber where it impacts the sample;
wherein a portion of part one of the beam that impacts the sample is absorbed by the sample;
wherein after that portion of part one of the beam is absorbed, the sample releases energy, producing an output absorbance wave;
wherein the non-absorbed portion of part one of the beam and output absorbance wave is then detected by the optical detector which creates data set 2, which is then sent to the data acquisition system;
wherein part two of the beam exits the beam splitter and enters the reference detector, which identifies the frequencies of part two of the beam, as well as the intensity of part two of the beam, creating data set 3, which is fed into the data acquisition system;
wherein the lock-in amplifier receives the various information including the initial frequency and intensity of the beam that exits the quantum cascading laser, which is data set 1, as well as data set 3 and data set 2, to then identify the unabsorbed portion of part one of the beam, identify the frequency and intensity of the output absorbance wave, determine and separate out that portion of the output absorbance wave that is the result of the blood plasma and water, and characterize the remaining frequencies and intensities of the output absorbance wave, the spectra;
wherein the continuous quantum cascading laser system is controlled by a computer; and
wherein a computer also receives the spectra from the and then conducts the comparison of the frequencies and intensities of the spectra with a database of known spectra to characterize the pathogenic microorganisms and other trace constituents in the sample.
